(19) United States
(12) Patent Application Publication
(10) Pub. No.: US 2004/0167404 A1
(43) Pub. Date: Aug. 26, 2004

(54) Characterization of middle ear effusion

(76) Inventor: Robert Bessler, Browns Point, WA (US)

Correspondence Address: CALFEE HALTER & GRISWOLD, LLP, 800 SUPERIOR AVENUE, SUITE 1400, CLEVELAND, OH, 44114, US

(21) Appl. No.: 10/783,136

(22) Filed: Feb. 20, 2004

Related U.S. Application Data
(60) Provisional application No. 60/448,611, filed on Feb. 20, 2003.

(51) Int. Cl. A61B 8/00

(52) U.S. Cl. 600/443

(57) ABSTRACT

A method for the detection and characterization of fluid behind the tympanic membrane. More specifically, a method for differentiating between infective and non infective effusions behind the tympanic membrane in animals, particularly humans, and more particularly, human children, based on analysis of the rheological properties of the fluid using an ultrasonic source.

### CROSS REFERENCE TO RELATED APPLICATION

This invention claims priority to U.S. Provisional Application No. 60/448,611, filed on Feb. 20, 2003, and is incorporated herein by reference in its entirety.

### BACKGROUND OF THE INVENTION

Otitis media is an inflammatory process in the middle ear and is the most common clinical condition seen by pediatricians in children fifteen years and younger. Otitis media (“OM”) is generally associated with the presence of a middle ear effusion and is considered a middle ear inflammation. Complications of undiagnosed OM can include hearing loss; left untreated, OM can lead to delays in the development of speech and language skills.

There are two key factors in the diagnosis of OM: detection of the presence of effusion; and characterization of the type of effusion as either serous, mucoid, or purulent. Decision by the health care provider regarding appropriate treatment hinges on confirmation of both the presence of effusion and its type—not all effusion types are treated the same. Health care practitioners use a variety of tests to evaluate a patient suspected of having OM. The only definitive tests for OM are myringotomy and tympanocentisis, procedures which involve direct aspiration of fluid from the middle ear, followed by visual and biochemical analysis of the fluid. These are invasive procedures performed in a surgical setting under anesthesia. Because they are invasive and have significant associated risks of complications, myringotomy and tympanocentisis are not used as standard diagnostic methods for OM.

Several other non-invasive diagnostic tests are available for evaluating OM, including acoustic reflectometry, tympanometry, pneumatic otoscopy, and otoscopy, however, none of these tests achieves 100% agreement with myringotomy or tympanocentisis; the overall likelihood of obtaining an accurate diagnosis using any of the non-invasive methods is no better than 50%. More importantly, the various non-invasive methods are useful only in identifying the presence of middle ear effusion; they provide no information regarding the type of effusion. Because of the risks associated with undiagnosed OM, and the recognized unreliability of the non-invasive diagnostic tests, patients who are diagnosed with middle ear effusions based on any of these non-invasive tests are often prescribed antibiotics. In many instances, these patients do not have OM. In addition to the increased cost burden of unnecessary antibiotic treatment, the patients are exposed to the side effects of antibiotics and the attendant and significant risk of developing antibiotic resistance. Accordingly, a more reliable, non-invasive method of diagnosing OM is needed.

### SUMMARY OF THE INVENTION

The present invention provides a method for the detection and characterization of fluid behind the tympanic membrane. The present invention also provides a method for differentiating between infective and non infective effusions behind the tympanic membrane in animals, particularly humans, and more particularly, human children, based on analysis of the rheological properties of the fluid, preferably using an ultrasonic source, preferably in the form of a probe embodied in a system and device that can be used in a health care provider's office.

This invention also provides a method for using a predetermined algorithm to correlate information collected by the ultrasound probe with a standard curve to confirm the presence of effusion. This invention also provides a method for analyzing the rheological properties of fluid behind the tympanic membrane, and correlating the rehological information about effusion with a standard curve for fluid viscosity. This invention also provides a method for identifying the type of effusion as serous, mucoid, or purulent based on viscosity. This invention also provides a method for determining the effusion type so as to provide advice on appropriate treatment protocols.

The present invention also provides a system for detecting and characterizing middle ear effusion comprising a probe, preferably an ultrasonic probe, which comprises at least one ultrasonic transducer. The probe operates in both transmit and receiver mode, and is insertable into an animal ear, more particularly a human ear, and most particularly a child's ear, for detecting and characterizing effusions behind the tympanic membrane. The system further comprises a power and control source in communication with the probe for transmitting and receiving the signal. The system further comprises a data collection source in communication with the probe for collecting signals, which is in communication with a data analysis source for analyzing the data. The system further comprises an algorithm for analyzing the data to provide rheological information which is in communication with the data collection and analysis sources, and an algorithm for correlating the data about rheological information with a standard curve to provide a measure of viscosity, and an algorithm for correlating information about viscosity to predetermined measures of viscosity for effusion type. Optionally, the system may further comprise an earpiece integrated with the probe containing encapsulated fluid or semi-fluid medium for signal transmission, or alternatively, a system for delivering and removing such medium to a patient's ear canal. Optionally, the system may also comprise an algorithm in communication with the data storage and analysis sources for determining treatment options based on analysis of effusion type. The system may be embodied in a small medical device, such as an ultrasonic device.

### DETAILED DESCRIPTION OF PREFERRED AND ALTERNATE EMBODIMENTS

Otitis media, an inflammatory process in the middle ear, is the most common clinical condition seen by pediatricians in children in the age range of fifteen years and younger. The presence of a middle ear effusion of any kind is considered a middle ear inflammation. Complications of undiagnosed OM can include hearing loss and consequently delay in development of speech and language skills. The combination of the gravity of the complications of undiagnosed OM and unsatisfactory diagnostic techniques often leads to unnecessary over-medication of a child with antibiotics. In addition to increasing the costs of treatment, such unnecessary treatment with antibiotics can lead to the development of antibiotic resistance.

The types of OM most frequently encountered include: (1) acute otitis media with effuision, which is characterized by the presence of fluid in the middle ear, and is accompanied by symptoms of middle ear inflammation in which the tympanic membrane is under positive pressure; (2) otitis media with residual effusion, which is characterized by the presence of fluid in the middle ear without symptoms of infection, in which patients may experience a “plugged ear” feeling and the tympanic membrane may be under negative pressure (retracted) or under neutral pressure; and (3) otitis media with effusion, which is characterized by the presence of asymptomatic middle ear effusion, without otoscopic signs of inflammation, that persists for more than 16 weeks.

Historic use of Ultrasound in Assessment of Middle Ear Effusion

There have been a few attempts to use ultrasound technology for detection of middle ear effusion; in each case, the investigators had varying success in the detection of effusion. But as with all other non-invasive detection methods, ultrasonographic methods provided no information regarding the type of ear effusion. Abramson et al used a 10 MHz non-focused transducer in A-mode inserted into an external ear canal to analyze the echo from the inner ear wall. In ears lacking effusion no echo was detected. For ears with effusion of any type an echo was clearly detectable. According to the methods of Abramson there was no ability to distinguish between types of effusion or to positively detect infection.

Alvord et al. used a gray-scale ophthalmic ultrasonic sector scanner (10 MHz, B scan) placed in the external auditory canal. Eleven subjects (7 normal, 4 with diagnosed middle ear effusion) were tested. The presence of fluid appeared on the ultrasonograms as a dark band positioned medial to the eardrum which was not seen on the ultrasonograms of ears lacking effusion. As with Abramson, according to the methods of Alvord there was no ability to distinguish between types of effusion or to positively detect infection.

An attempt to correlate the results of myringotomy with endoluminal ultrasonography was done by Wu et al. The investigators performed ultrasonographic scans of healthy subjects and patients with OM using a 20 MHz imaging transducer (B scan) mounted on a 6 French catheter. The Wu study included six children diagnosed with OM using noninvasive techniques who had been treated unsuccessfully for 3 months and thereafter scheduled for myringotomy. The transducer used in this study was perpendicular to the ear canal axis allowing only for lateral scans. The results were considered positive if signals were recorded behind the plane of tympanic membrane. Presence of fluid was successfully detected in five cases based on the confirmed presence of fluid by myringotomy. In one of six patients, the sonogram failed to detect fluid. Again, there was no ability to characterized the viscosity or type of effusion using the ultrasound technique of Wu.

Use of Ultrasound in Detecting and Characterizing Middle Ear Effusion

According to the methods of the present invention, information is obtained regarding both the presence and type of ear effusion. In one embodiment, these methods involve using an ultrasound probe suitable for pediatric patients in a hand-held device, whereby an ultrasound signal is directed into the middle ear and the echo shows distinct differences in cases of middle ear effusion versus no fluid. These methods also characterize the viscosity of effusion fluid by analysis of the rheological properties of the fluid and then correlate this information with disease state based on standards for effusion viscosity. With viscosity information, the methods of this invention then provide for the characterization of the effusion based on type. Provided with information about effusion type, a health care provider can decide the most appropriate treatment course for a patient. The procedure is non-invasive and painless and is at least 97% effective for detecting middle ear effusion and characterizing effusion viscosity.

Effusion Detection and Characterization System using an Ultrasonic Probe

This invention provides a diagnostic system for detection and characterization of middle ear effusion in the typical setting of a general pediatrician's office with conscious patients. With this system, a health care provider can collect information, preferably in the form of ultrasonic scans, on conscious patients to determine presence and type of middle ear effusion based on the signal analysis performed by the software-embodied algorithms working with the system.

The probe is preferably adapted to accommodate the size limitations of the pediatric ear canal (minimum diameter of about 3 mm and preferred maximum diameter of about 6 mm), with consideration of the curved geometry defined by the cartilagenous structures lining the outer half of the outer canal. Preferably, a curved array ultrasonic probe is used, comprising at least one miniature transducers arranged in an array such that each transducer is aimed at different angle with overlapping fields of view to ensure that the tympanic membrane is fully covered by the ultrasonic beams radiated from the transducer array. The array may be a single transducer that is moved mechanically so as to scan the tympanic membrane, or may be made up of multiple transducers that scan discretely or in a phased pattern.

Scanning transducer length is from about 2 to about 10 mm, preferably from about 2 to about 5 mm and most preferably about 3.2 mm. The diameter for the transducer and steering mechanism is preferably from about 1 to about 5 mm and most preferably about 1.2 mm.

The transducer is preferably operated in A-mode. In A mode, the measured values are the amplitude of the echo and elapsed time between original pulse and echo reflected from the target. The distance to the target and echo elapsed time are related by simple formula. The advantage of A-mode is that it gives positional information with relatively simple instrumentation. Generally, in A mode, the same transducer is used as the signal source and receiver in transmission-detection regime.

The amplitude of an echo pulse reflected from the boundary between two different media depends on the values of characteristic impedance of both media. The acoustic impedance of air is 0.0004 MRayl while for water it is 1.48 MRayl. The tissue acoustic impedance is similar to impedance water (for example 1.18 MRayl fat, 1.56 MRayl liver). The reflection at the interface of two media having substantially different characteristic impedance (such as air and water), is significant. In the case of a water-air interface the reflection is in the range of 99%.

The ultrasound-transmitting medium between the transducer and tympanic membrane may take various forms, such as water, gel, or other appropriate conductive medium. The medium is preferably contained within a delivery system that permits delivery and removal of the medium from the ear canal. The delivery system may comprise a separate medium delivery and evacuation means. Alternately, the delivery system may be encapsulated and contain the medium in an enclosed vessel insertable in the ear canal.

The device also comprises an ultrasonic pulser/receiver which provides a series of pulses activating transducer for amplifying the echo coming form the middle ear cavity, a data storage module, such as any standard recording medium, a data processing module for signal processing and comprising Theological characterizing and diagnostic algorithms, and optionally a visual display. Additional components such as data storage and processing media, and view screen may be separate from the system, in connection with the system, or miniaturized and incorporated into a hand-held component, and may also comprise transducer control comprising a steering mechanism for guiding or directing the transducer to desired area of the tympanic membrane for scanning (tip of the transducer should be positioned approximately 10 mm from the tympanic membrane). The components may also comprise a thermostatic control to heat the water to physiological temperature for patient comfort.

Differentiation of Ear Effusions

The methods of this invention permit the differentiation between serous (thin) and mucoid (thick) and purulent effusion fluid by analysis of the ultrasonic signal attenuation of the echo signal reflected from the back of the inner ear. The attenuation is determined, for example, by comparing power spectra obtained by Fourier transformation of the original and reflected pulse. The center frequency shift resulting from attenuation of the echo is correlated with the viscosity of the fluid.

An ultrasound pulse is generated by the miniature transducer inserted into the auditory canal of the ear. The canal will is filled with water. Since the characteristic acoustic impedance of water (Z_{w}) is close to the impedance of the tympanic membrane (Zₜ) filling the channel with water minimizes reflection of the pulse from the front surface (proximal) of the membrane (low reflectance coefficient). The pulse reaches the interface between the back of the membrane and the inner ear cavity. For a healthy ear, the inner ear is filled with air. The acoustic impedance of air (Zₐ) is significantly lower than impedance of tissue (Z_{t.}). The reflectance coefficient of a tissue-air interface is high. Therefore this interface will produce a strong echo, which will be detected by the transducer. The echo signal will consist of multiple reflections arising between the membrane and front face of the transducer. These secondary signals will be eliminated by time windowing. For Zₐ=0.0004 MRayl and Zₜ=1.62 MRayl, the reflection coefficient of the tissue-air interface exceeds 0.99.

In the case of otitis media, the inner ear is filled with fluid. The presence of fluid significantly decreases the reflection coefficient on the interface between the back (distal) plane of the tympanic membrane and the inner ear cavity. This allows the pulses to penetrate the inner ear cavity until they reach the back of the inner ear. This is the interface between fluid and bone.

The acoustic impedance of bone is approximately four times higher than air. Therefore this interface will produce an echo signal, which will be detected by the transducer. Assuming that the fluid impedance is same as for water (Z_{f}=1.48 MRayl) and Z_{b}=6.00 MRayl, the reflection coefficient of the fluid-bone interface is 0.6.

The length of the inner ear is significantly larger then the thickness of the tympanic membrane. Therefore the echo from the membrane-air interface (in the case of ear without effusion) can easily be distinguished from the echo generated by the reflection from the back wall of the inner ear (in the case of ear with effusion). This is done by time windowing. Assuming that the sound speed in the fluid is 1500 m/s (blood 1550 m/s), and that the distance between the tympanic membrane and, the back of inner ear is 10 mm, the difference between echoes from the membrane-air interface and from the back of inner ear will be in the range of 13.2 μs. Selective time-domain gating of pulses will allow distinguishing respective echo sources based on the elapsed time.

The differentiation of fluid viscosity (between low and high viscosity) is done based on a spectral analysis of the signal reflected from the rear wall of the inner ear compartment (fluid-bone interface).

A series of artificial effusion fluids is used in a mock setting to create a standard curve covering the viscosity range from 0.5 poise to 20 poise. The echoes reflected from the inner ear wall are digitally recorded and analyzed using, for example, Fast Fourier Transform software. Analysis of the frequency shift is performed by first digitally collecting the data from the transducer. The recorded signal is then exported for frequency analysis.

The peak frequencies are then examined for each of the data sets, and a shift in this peak are used as a measure of viscosity of middle ear effusion fluid to create a standard curve.

Diagnosis of OM is ultimately achieved by correlating the results of ultrasonography conducted by the present methods with established standards for identifying disease state. Once data is obtained about the viscosity of fluid detected in a patient's ear, including a measure of whether the fluid is serous, mucoid, or purulent, the health care provider may then tailor an appropriate treatment plan, which may or may not include antibiotics. Optionally, the devices provided by this invention include algorithms that provide a diagnostic reading to health care providers based on effusion presence and viscosity.

### EXAMPLE 1

In vitro Testing in an Artificial Ear Model

A series of artificial effusion fluids was prepared covering the viscosity range from 2 poise to 20 poise. The echoes reflected from the inner ear wall were digitally recorded and analyzed.

A miniature ultrasonic testing cell was constructed [results of study executed by Biomec using testing cell by Katz and Hazony, unpublished study], which consists of a broad band transducer incorporated into a titanium probe. The transducer was permanently attached to the 12 mm long and 5 mm diameter titanium bar. A 1.5-mm slot was cut at the end of the titanium bar creating a test chamber. The transducer was activated with 10 ns sharp electric signals, which generate a 50 ns acoustic pulse.

The study was conducted using a physical ear model with the series of artificial effusion fluids. Saline solution containing mucin from porcine stomach and bovine albumin (Sigma) was used to simulate the middle ear effusion. The solutions were prepared in phosphate buffered saline (PBS). The concentration of mucin in the middle ear effusion varies from 8 to 25% of the non-dialyzable components of the effusion fluid. The total amount of nondialysable components is in the ranges of 355 to 574 μg/ml. Albumin content was found to be between 119 to 156 μg/mg of non-dialyzable components. The concentration of mucin varies from 2.84 mg/dl to 14.35 mg/dl and the concentration of albumin varies from 4.88 mg/dl to 7.89 mg/dl (assuming an average of 138 μg/mg of non-dialyzable components). Since mucin was found to be the dominating factor determining viscosity of middle ear effusion, the albumin concentration in “artificial middle ear effusion” was maintained constant at 6.4 mg/dl. The viscosity of “artificial middle ear effusion” was adjusted by changing the concentration of mucin.

The transducer was operated in transmitter-receiver mode such that the same transducer was used for pulse generation and detection of echoes in A-mode: unfocused; cylindrical shape of 1.8 mm in diameter; body made of titanium; central frequency of 20 MHz; frequency of pulse repetition of 100 Hz; tissue wavelength of 77 um; bandwidth (6 dB) of 7 MHz; beam width of 4.9. The transducer was mounted on the tip of hypodermic tubing 130 mm long, 1.8 mm diameter. The ultrasonic transducer connected pulser-receiver (to Panametric Model 5072PR). Sharp 10 ns electric pulses were applied to the transducer, which generates 50 ns acoustic signals along the longitudinal axis. The echoes generated on the interface were received by the same transducer in the silent period and were converted to an electric signal. The signal exiting the transducer and the echo were captured with a digital oscilloscope (Tektronix) with bandwidth 0.5 GHz. The signals were digitally stored for analysis and further processing.

Results

The in vitro study demonstrated that in the presence of fluid behind the membrane 6 fold decrease of the echo amplitude at center frequency, allowing for high accuracy detection of the fluid presence behind the membrane. The viscosity of the artificial effusion fluid was be distinguished in the expected viscosity range between 2 cSt and 20 cSt, allowing for the determination of type of the fluid according to presently used three-point clinical scale (serous, purulent, mucoid). The sensitivity to fluid detection is not influenced by the membrane thickness within the tested range.

### EXAMPLE 2

Clinical Evaluation

Children scheduled to undergo bilateral myringotomy with pressure equalizaion tube placement (BMT) were assessed. Each patient was anesthetized for the purposes of the tube placement surgery. At the time of surgery, the patient's ear was examined under the operating microscope as per usual routine for BMT. Any wax or debris in the external auditory canal was cleaned out. Otoscopic evaluation of the ear was done by the operating surgeon. The following results of the otoscopic evaluation were recorded: color change of tympanic membrane (redness), translucency, discharge, visual detection of effusion, resting position of tympanum (retracted, neutral). In the case when membrane perforation was observed, the ultrasonic study was not performed.

The following descriptive three-point scale is used to characterize fluid: (1) thin serous, (2) thick mucoid, and (3) thick purulent.

The patient was subjected to the routine testing that precedes surgical tube placement surgery, including typanometric evaluation of the ear. The ultrasonic detector was tested on anesthetized patients prior to tube placement surgery. The transducer was mounted on the Frazier suction (5 French) probe for insertion into the auditory canal. Prior to use, the transducer was sterilized.

The auditory canal of the patient was filled with 1 ml of warm, sterile water. The surgeon otoscopically monitored the insertion of the transducer. The transducer was rested along the canal wall to stabilize its position in close proximity of the tympanic membrane, with no direct contact with the membrane. The transducer was aimed at the central portion of the drum underneath the umbo. The series of pulses was applied to the transducer and echo signals and the reference pulse was digitally recorded for further processing. After scanning was completed, the water was removed with suction.

The patient underwent routine tube placement surgery, which include withdrawal of middle ear effusion. The doctor visually evaluated the appearance and rheological properties of the fluids and he classified fluid appearance according to the three level scale. Fluid samples were also sent for culture. Since there is no official classification for fluid properties, an arbitrary three-point scale was used to describe the fluid properties. The results of fluid viscosity were correlated with the spectral analysis of the echo signals.

This three point scale was designed so that about one third of the children should fall into each of the three ranks. We tested for a correlation between the fluid viscosity as measured by the device of the present invention and the fluid viscosity evaluation of the surgeon.

The following results were obtained: there was virtually 100% agreement between the results of ultrasonographic testing and myringotomy. The ultrasonic probe detected the presence of fluid in each case and accurately characterized the type of fluid based on viscosity/rheological properties.

1. A diagnostic test for otitis media, comprising: detecting the presence and measuring the viscosity of middle ear effusion in a human patient; and comparing the measured viscosity of the middle ear effusion in the human patient with at least three predetermined values for effusion viscosity, wherein such comparison provides information regarding the likelihood of presence of bacterial infection in the middle ear effusion in the human patient.

2. The diagnostic test of claim 1 wherein each of said predetermined values is based on a plurality of predetermined ranges of fluid viscosity measurements.

3. The diagnostic test of claim 2 wherein the predetermined ranges of fluid viscosity measurements are obtained from fluid viscosity measurements selected from the group consisting of middle ear effusions from the general human population, middle ear effusions from a select population of human subjects, and simulated middle ear effusions from a model system, and wherein said comparing step comprises determining in which of said plurality of predetermined fluid viscosity ranges the human patient's middle ear effusion viscosity falls.

4. A method for detecting in an animal the presence and characterizing the viscosity of middle ear effusion by transmitting a signal into an ear canal of the animal, receiving a reflection of the signal, and comparing the received signal with a standard comprising a range of signals obtained with fluids of varying viscosities, wherein the range of signals are normalized to reflect a measurement of viscosity.

5. The method according to claim 4 wherein at least one ultrasound transducer is used for signal transmission and reception.

6. A method for determining if a human patient is a candidate for receiving antibiotic treatment, wherein the presence of middle ear effusion in the patient is detected and the effusion viscosity is determined and compared with at least one predetermined fluid viscosity value.

7. The method of claim 6 wherein an ultrasound probe is used to detect and measure effusion viscosity.
